(19) United States
(12) Patent Application Publication
(10) Pub. No.: US 2025/0361278 A1
(43) Pub. Date: Nov. 27, 2025

(54) RECOMBINANT PRODUCTION OF GROWTH FACTORS IN ALGAE FOR CELL CULTURE APPLICATIONS

(71) Applicant: Phycin Inc., Frederick, MD (US)

(72) Inventor: Jun Wang, Frederick, MD (US)

(73) Assignee: Phycin Inc., Frederick, MD (US)

(21) Appl. No.: 18/674,768

(22) Filed: May 24, 2024

(51) Int. Cl. C07K 14/485; C07K 14/495; C07K 14/50; C12N 1/12; C12N 5/00; C12N 15/82; C12R 1/89

(52) U.S. Cl. CPC C07K 14/485; C07K 14/495; C07K 14/501; C07K 14/503; C12N 1/125; C12N 5/0018; C12N 15/8251; C12N 2501/00; C12N 2501/117; C12N 2501/119; C12N 2501/12; C12N 2501/13; C12N 2501/135; C12N 2501/148; C12N 2501/15; C12N 2501/155; C12N 2501/165; C12N 2501/22; C12N 2501/2301; C12N 2501/2302; C12N 2501/2304; C12N 2501/2305; C12N 2501/2306; C12N 2501/2307; C12N 2501/2308; C12N 2501/231; C12N 2501/2313; C12N 2501/2315; C12N 2501/2318; C12N 2501/232; C12N 2501/355; C12R 2001/89

(57) ABSTRACT

The present invention provides a method for producing recombinant growth factors using an algal expression system, which offers advantages over traditional platforms, and the algae-derived growth factors can be used to formulate cell culture media for mammalian cells without the risk of pathogen contamination or endotoxins.

### FIELD OF THE INVENTION

The present invention relates to the field of recombinant protein production. More specifically, it relates to methods for producing growth factors in algae for use in cell culture applications.

### BACKGROUND

Epidermal growth factor (EGF), acidic fibroblast growth factor (aFGF), and basic fibroblast growth factor (bFGF) are important signaling molecules that stimulate cell proliferation and differentiation in various tissues.

Current production methods for recombinant growth factors, including EGF, aFGF and bFGF rely on mammalian cells, bacteria, yeast and higher plants. However, these systems have drawbacks in terms of high production costs, safety concerns, and scalability issues. There is a need for alternative production platforms that can provide these growth factors in a cost-effective, safe, and scalable manner.

Mammalian and bacterial platforms encounter several challenges in meeting the demand for growth factors:
Risk of pathogens: Growth factors derived from mammalian cell lines (e.g., CHO and HEK293) carry the potential risk of infectious human and animal agents. Contaminations by pathogenic microbes pose severe health threats to patients.
Undesirable endotoxins: Commercially available growth factor products often contain variable levels of endotoxins, even if they fall within the currently accepted low limits (up to ˜1 EU/μg). Such endotoxins can significantly impact cell viability, behavior, and function.
Compromised activity: Many growth factors, despite being small proteins, possess complex structures. For instance, Transforming Growth Factor β-1 (TGF β-1) is a homodimer with a disulfide bond and three additional intramolecular disulfide bonds. E. coli cells lack the capability to assemble proteins of this complexity, necessitating additional steps like re-folding and homo-dimerization for TGF produced in E. coli.
High cost: Mammalian systems are capable of producing high-quality growth factors with efficient refolding and dimerization processes. However, due to the potential lethality of growth factors as “messenger” molecules when over-expressed, they must be maintained at safe concentration levels within mammalian host cells. This low expression, typically at the milligram per liter level, results in high prices for commercial growth factors, often exceeding $1,000 per milligram.

Algae present a promising alternative as a production host for recombinant proteins. They offer advantages in terms of biosafety, scalability, lack of toxic contaminants, and oral delivery potential. The transgenes for growth factors can be expressed from either chloroplast genome or nuclear genome.

### SUMMARY OF THE INVENTION

The present invention addresses the critical needs for affordable, animal component and endotoxin free growth factors by providing a novel method of producing growth factors using an algae-based expression system. The growth factors are expressed from the nuclear or chloroplast genome of the algae, either monocistronically or polycistronically.

The algae-based platform addresses the challenges of current production methods by offering a more efficient, cost-effective, and safe approach for producing growth factors. It eliminates the risk of pathogen contamination, avoids undesirable endotoxins, and enables the proper assembly of complex growth factor structures.

In one aspect, the invention provides expression vectors comprising nucleotide sequences encoding EGF, aFGF, bFGF or TGF superfamily proteins operably linked to promoters and regulatory elements for expression in algae. The vectors may optionally include selectable markers. Transgenic algae are generated by introducing the vectors via methods such as glass bead agitation, electroporation, biolistic bombardment or Agrobacterium-mediated transformation.

In another aspect, artificial intelligence-assisted molecular design is used to enhance the thermostability of growth factors while maintaining their specific activity. This enables the development of novel stem cell culture protocols that require less frequent medium changes, reducing sample handling and potential contamination risks.

In yet another aspect, the algae-produced growth factors are used in mammalian and stem cell culture media. The growth factors can be used individually or in combination, in purified form or as algal extracts. The resulting culture media are free of human/animal pathogens and bacterial endotoxins.

The advantages of the present invention include high production yield, great scalability, enhanced biosafety and low cost compared to current production systems. The algae-based platform enables large-scale production of recombinant growth factors for cell culture applications.

### BRIEF DESCRIPTION OF THE DRAWINGS

FIG. 1 shows an expression vector for producing human EGF from the chloroplast genome.

FIG. 2 shows an expression vector for polycistronic production of human aFGF from the chloroplast genome.

FIG. 3 shows an expression vector for secretory production of bFGF from the nuclear genome.

FIGS. 4-6 show data demonstrating that algae-produced aFGF promotes growth of human induced pluripotent stem cells comparably to bFGF without affecting pluripotency marker expression.

FIG. 7 compares the performance equivalence between algal-derived wildtype bFGF and commercial bFGF in stimulating the proliferation of Mesenchymal Stem Cells (MSCs).

FIG. 8 demonstrates the improved stability of bFGF when applied to Induced Human Pluripotent Stem Cells (iPSCs) and the heat treatment experiment on rat fibroblast cells.

FIGS. 9A, 9B, and 9C show that the engineered bFGF exhibits significant thermostability, retaining over 50% of its activity even after three days of pre-treatment at 37° C. In contrast, wildtype bFGF loses its entire activity under the same conditions.

FIG. 10 emphasizes the detrimental impact of endotoxins on the health of animal cells during culture and the lack of endotoxins in the algae-derived growth factors produced by the claimed method of this invention.

FIG. 11 shows the successful recombinant expression of TGF superfamily proteins from the algal nuclear genome.

### DETAILED DESCRIPTION

The present invention provides methods for producing recombinant growth factors in algae. The general methodology involves: 1) constructing expression vectors encoding growth factors; 2) introducing the vectors into an algal host; 3) expressing the protein from the algal nuclear or chloroplast genome; and 4) purifying the recombinant protein.

Any protein or peptide that can be encoded by a DNA sequence can be expressed using this system. Suitable algal hosts include, but are not limited to, Chlamydomonas reinhardtii, Nannochloropsis sp., Dunaliella sp. and Haematococcus pluvialis. The gene of interest can be a natural sequence or a synthetic, codon-optimized sequence. Expression can be monocistronically or polycistronically from the chloroplast genome.

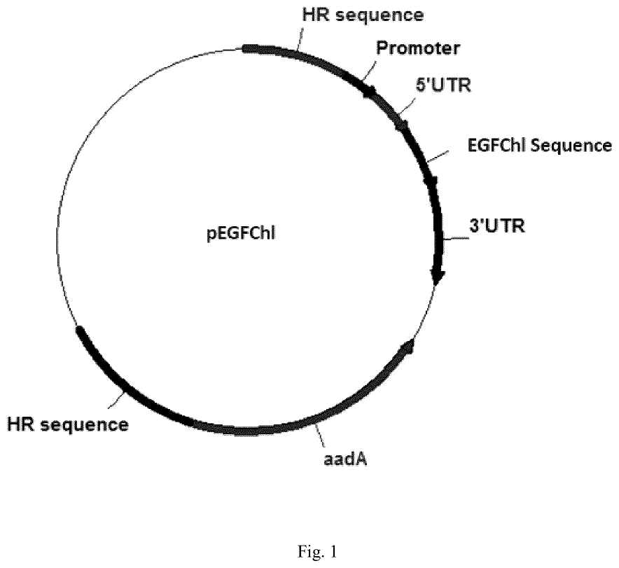

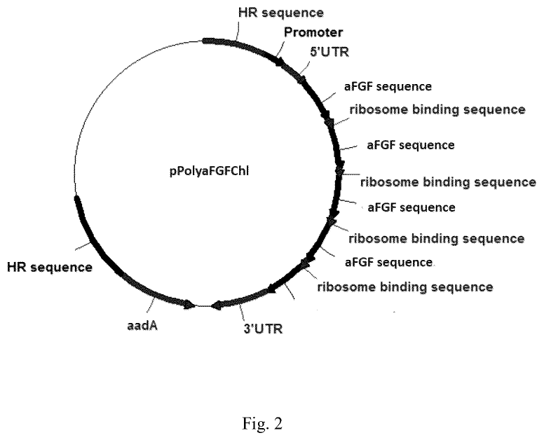

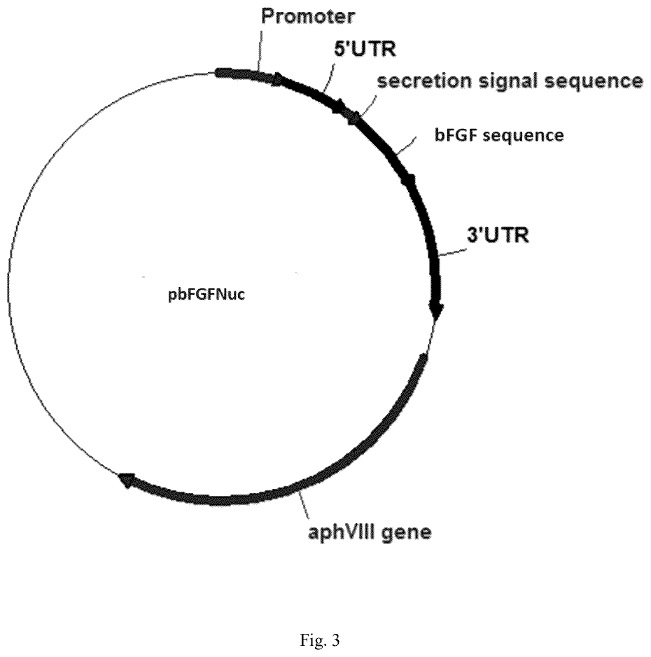

Specific examples are provided for producing PTD-fused human EGF, aFGF, bFGF and TGF superfamily proteins. EGF is expressed monocistronically from the chloroplast genome (FIG. 1). aFGF is expressed polycistronically from the chloroplast genome using repeated coding sequences separated by ribosome binding sites (FIG. 2). bFGF is expressed from the nuclear genome and secreted into the culture medium using a native secretion signal (FIG. 3). Selectable markers such as aadA and aph VIII are included for generating stable transgenic lines.

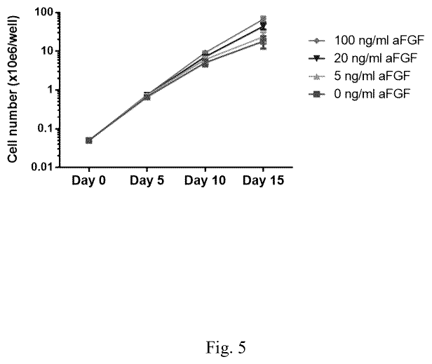

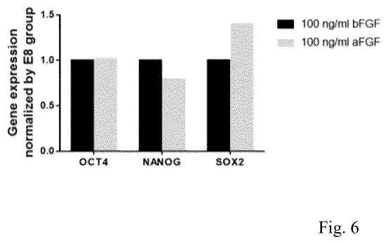

Artificial intelligence-assisted molecular design is used to enhance the thermostability of bFGF while maintaining its specific activity. Through molecular dynamics simulations and experimental validation, specific residues and regions contributing to protein flexibility are identified. The resulting bFGF variants exhibit improved thermostability, enabling the development of novel stem cell culture protocols that require less frequent medium changes (FIGS. 5-6).

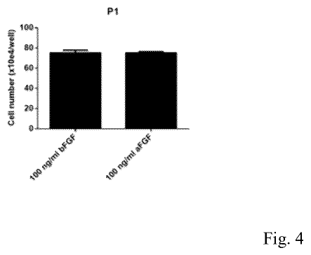

The algae-produced growth factors can be further engineered to enhance membrane permeability and thermostability. They can be used individually or in combination in mammalian cell culture media at desired concentrations. Data is presented showing that algae-produced growth factors promote the growth of human induced pluripotent stem cells and mesenchymal stem cells comparably to commercial products (FIGS. 4-5).

The advantages of the algae-based system include great scalability, lack of pathogen and endotoxin contamination, high yields from polycistronic chloroplast expression, and cost-effectiveness. It provides a novel platform for the large-scale production of recombinant growth factors for cosmetic and cell culture applications.

### EXAMPLES

### Example 1: Production of EGF in Chlamydomonas reinhardtii Chloroplast

The coding sequence for human EGF was cloned into the expression vector pEGFCh1 (FIG. 1). The vector contains the aadA gene for spectinomycin resistance. C. reinhardtii cells were transformed with pEGFCh1 by biolistic bombardment and transgenic lines were selected on spectinomycin-containing media. Integration of the transgene into the chloroplast genome was confirmed by PCR. EGF was expressed monocistronically under the control of chloroplast regulatory elements. The recombinant protein was purified from the algal biomass by chromatography methods.

### Example 2: Production of aFGF in Chlamydomonas reinhardtii Chloroplast

The coding sequence for human aFGF was cloned into the expression vector paFGFCh1 (FIG. 2). The vector contains four repeats of the aFGF sequence separated by chloroplast ribosome binding sites for polycistronic expression. The aadA gene was included for selection. Chlamydomonas cells were transformed with paFGFCh1 by electroporation. Transgenic lines were selected and confirmed as in Example 1. aFGF was purified from the algal biomass.

### Example 3: Production of Secreted bFGF in Chlamydomonas reinhardtii Nucleus

The coding sequence for human bFGF and a native secretion signal was cloned into the expression vector pbFGFNuc (FIG. 3). The vector contains the aph VIII gene for paromomycin resistance. C. reinhardtii cells were transformed with pbFGFNuc by glass bead agitation. Transgenic lines were selected on paromomycin-containing media and confirmed by PCR. bFGF was secreted into the culture medium and purified by chromatography.

### Example 4: Use of Algae-Produced aFGF in Human Stem Cell Culture

Algae-produced aFGF from Example 2 was tested for its ability to support the growth of human induced pluripotent stem cells (hiPSCs). hiPSCs were cultured in E8 medium supplemented with either bFGF (control) or aFGF at different concentrations. After 4 days of culture, aFGF and bFGF resulted in similar cell growth (FIG. 4). Over multiple passages, aFGF at 100 ng/ml gave the highest hiPSC yield (FIG. 5). RT-PCR analysis showed that aFGF-cultured hiPSCs expressed the pluripotency markers Oct4, Nanog and Sox2 at levels comparable to bFGF-cultured cells (FIG. 6). This demonstrates that algae-produced aFGF can be used as an alternative to mammalian bFGF in stem cell culture media.

### Example 5: Production of TGF Superfamily Proteins from Chlamydomonas Nuclear Genome

The coding sequences for human TGF-β1, BMP-2 and BMP-7 were cloned into algal nuclear expression vectors. Chlamydomonas reinhardtii cells were transformed and transgenic lines were selected.

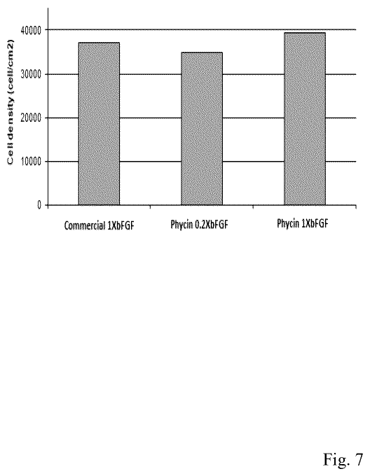

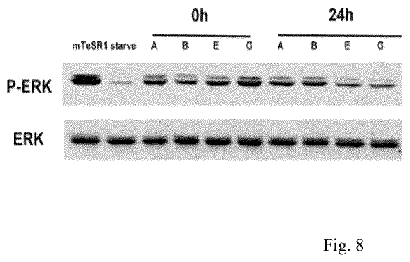

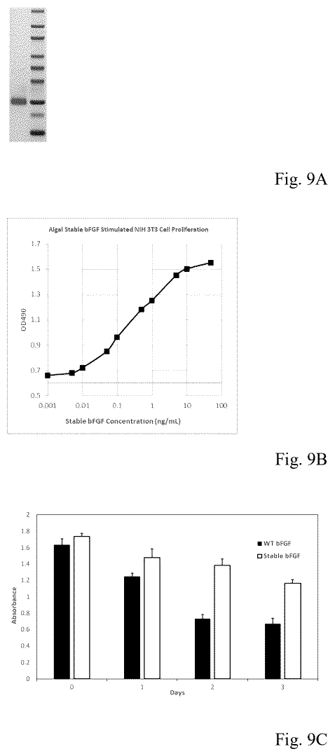

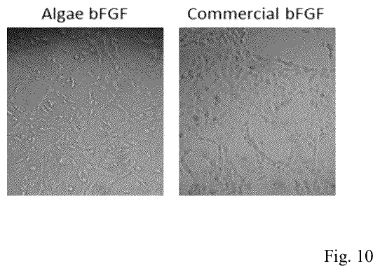

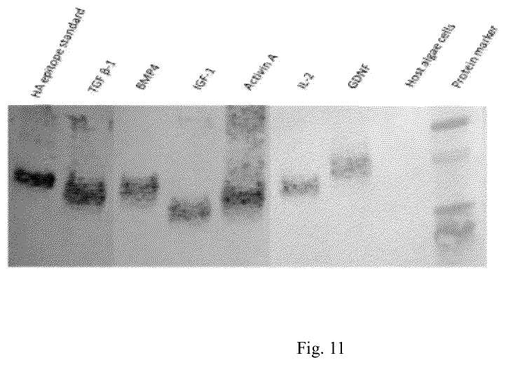

FIG. 7 compares the performance equivalence between algal-derived wildtype bFGF with commercial bFGF in stimulating the proliferation of Mesenchymal Stem Cells (MSCs). FIG. 8 demonstrates the improved stability of bFGF when applied to Induced Human Pluripotent Stem Cells (iPSCs), similar to the heat treatment experiment on rat fibroblast cells displayed in FIG. 9A-C. The engineered ebFGF exhibits significant thermostability, retaining over 50% of its activity even after three days of pre-treatment at 37° C. In contrast, wildtype bFGF loses its entire activity under the same conditions. Moreover, FIG. 10 emphasizes the detrimental impact of endotoxins on the health of animal cells during culture. The algae-derived growth factors produced from algae using the claimed method of this invention lack endotoxins and support distinct cell morphology compared to bacterial-derived counterpart. The colorimetric western blot result of FIG. 11 shows the representative complex growth factors produced by the method of this invention in algae. Primary antibody against HA epitope was used.

The above examples illustrate the production and application of algae-derived growth factors. The products are free of contaminating pathogens and endotoxins, making them safe and reliable for cosmetic and cell culture uses. The expression vectors and methods are applicable to producing other growth factors and cytokines of interest from algae.

All publications, patents, and patent applications cited herein are incorporated by reference in their entirety as if each individual publication, patent or patent application were specifically and individually indicated to be incorporated by reference. The citation of any publication is for its disclosure prior to the filing date and should not be construed as an admission that the present invention is not entitled to antedate such publication by virtue of prior invention.

It should be understood that the foregoing relates only to the exemplary embodiments of the present invention and that numerous changes may be made therein without departing from the scope of the invention as defined by the following claims.

1. A method of producing growth factors in an algal expression host, comprising: (a) constructing an expression vector comprising a nucleotide sequence encoding a growth factor selected from the group consisting of EGF, aFGF, bFGF and TGF superfamily proteins, operably linked to regulatory elements for expression in algae; (b) introducing said vector into an algal host cell to generate a transgenic algal line; (c) expressing the growth factor from the algal nuclear or chloroplast genome; and (d) purifying the recombinant growth factor from the algal biomass or culture medium.

2. The method of claim 1, wherein the algal host is selected from the group consisting of Chlamydomonas reinhardtii, Nannochloropsis sp., Dunaliella sp. and Haematococcus pluvialis.

3. The method of claim 1 or claim 2, wherein the growth factor is expressed from the chloroplast genome monocistronically or polycistronically.

4. The method of any one of claims 1-3, wherein the nucleotide sequence is a natural sequence or a synthetic codon-optimized sequence.

5. The method of any one of claims 1-4, wherein the vector further comprises a selectable marker gene.

6. The method of any one of claims 1-5, wherein the growth factor is of human, porcine, rat, mouse, feline, canine or equine origin.

7. The method of any one of claims 1-6, wherein artificial intelligence-assisted molecular design is used to enhance thermostability of the growth factor while maintaining its specific activity.

8. A cell culture medium for proliferation and/or differentiation of mammalian cells, comprising a recombinant growth factor produced by the method of any one of claims 1-7.

9. The cell culture medium of claim 8, comprising two or more different growth factors produced by the method of any one of claims 1-7.

10. The cell culture medium of claim 8, further comprising one or more components selected from the group consisting of VEGF, PDGF, FGF-4, FGF-6, TGFs-b, TGF-a, Epo, IGF-I, IGF-II, IL-1, IL-2, IL-4, IL-5, IL-6, IL-7, IL-8, IL-10, IL-13, IL-15, IL-18, IL-20, TNF-a, TNF-b, INF-g, G-CSF, GM-CSF, M-CSF, PLGF, NGF, KGF, BMP-4, HGF, leptin, noggin, and thymosin beta 4.

11. The cell culture medium of claim 9, wherein the mammalian cells are stem cells, fibroblasts, keratinocytes or endothelial cells.
